(19) United States
(12) Patent Application Publication
(10) Pub. No.: US 2017/0020423 A1
(43) Pub. Date: Jan. 26, 2017

(54) BIOLOGICAL INFORMATION MEASURING DEVICE AND DRUG SOLUTION SUPPLY DEVICE

(71) Applicant: Seiko Epson Corporation, Tokyo (JP)

(72) Inventors: Tetsuji FUJITA, Chino (JP); Kuniaki TANAKA, Matsumoto (JP); Makoto KATASE, Azumino (JP); Yoshihiko MOMOSE, Shiojiri (JP)

(21) Appl. No.: 15/205,435

(22) Filed: Jul. 8, 2016

(30) Foreign Application Priority Data
Jul. 23, 2015 (JP) 2015-145573

(51) Int. Cl. A61B 5/1486; A61B 5/00; A61M 5/142; A61M 5/148; A61B 5/145; A61M 5/172

(52) U.S. Cl. CPC A61B 5/14865; A61B 5/14532; A61M 5/1723; A61M 5/142; A61M 5/148; A61B 5/742; A61B 2562/0209; A61M 2202/07; A61M 2230/005; A61M 2230/201

(57) ABSTRACT

An information measuring device includes a detection film placed on the surface of a first needle section, an electrically conductive second needle section, and an electric current detection circuit which detects an electric current between the detection film and the second needle section. The major axis of the first needle section is 0.1 mm or more and 0.3 mm or less.

### BACKGROUND

1. Technical Field

The present invention relates to a biological information measuring device and a drug solution supply device.

2. Related Art

Diabetic patients are divided into type I and type II according to the symptoms, and in both types, insulin secretion from the pancreas is not normal. In particular, in type I diabetic patients, insulin is not secreted at all due to a pancreatic disease. Then, blood collection is performed several times a day including 4 times, before each meal and before going to bed, and the blood glucose level is measured. When the blood glucose level is high, insulin is administered.

A continuous glucose monitoring device which continuously and automatically measures the blood glucose level in such a patient has been disclosed in JP-T-2010-537732 (PTL 1). The continuous glucose monitoring device is abbreviated as “CGM”. In this CGM, an enzymatic reaction is utilized in the measurement of a glucose level. As the enzyme, glucose oxidase is used. When glucose and oxygen are present in the vicinity of the enzyme, gluconic acid and hydrogen peroxide are generated. The amount of hydrogen peroxide is detected by measuring the amount of an electric current generated by electrolysis of the generated hydrogen peroxide. Based on this, a glucose level is calculated.

In PTL 1, a cylindrical needle was used. Then, in the needle, a substrate was placed, and on the substrate, a plurality of electrodes were placed. After the needle was inserted into the skin tissue of a patient, the substrate was left in the skin tissue and the needle was pulled out. By doing this, the substrate was left in the skin tissue and exposed therein. Since the substrate was disposed in the inside of the needle to be inserted into the skin tissue, the outer diameter of the needle was large. Therefore, when the needle was inserted into the skin tissue, the needle gave pain to the patient. Due to this, a biological information measuring device which gives less pain to a patient when a needle is inserted into the skin tissue has been demanded.

### SUMMARY

An advantage of some aspects of the invention is to solve the problems described above and the invention can be implemented as the following forms or application examples.

### Application Example 1

A biological information measuring device according to this application example includes a first sensor electrode which is inserted into a body by piercing the skin surface, a second sensor electrode which is inserted into the body by piercing the skin surface, and an electric current detection section which detects an electric current between the first sensor electrode and the second sensor electrode, and measures in-vivo information.

According to this application example, the biological information measuring device includes a first sensor electrode, a second sensor electrode, and an electric current detection section. The electric current detection section detects an electric current flowing between the first sensor electrode and the second sensor electrode. Then, by detecting the electric current, the in-vivo information is measured.

The first sensor electrode and the second sensor electrode are separated, and the first sensor electrode is inserted into a first insertion place. The second sensor electrode is inserted into a second insertion place. Therefore, as compared with the case where a needle internally including a substrate having the first sensor electrode and the second sensor electrode placed thereon is used, the major axes of the first sensor electrode and the second sensor electrode to be inserted into the skin can be made short. As the major axes of the first sensor electrode and the second sensor electrode are shorter, a pain spot of a subject is less stimulated. Therefore, the biological information measuring device of this application example enables the subject to feel less pain even when the first sensor electrode and the second sensor electrode are inserted into the subject.

### Application Example 2

In the biological information measuring device according to the application example, it is preferred that the major axis of the first sensor electrode is 0.1 mm or more and 0.3 mm or less.

According to this application example, the major axis of the first sensor electrode is 0.1 mm or more. The major axis refers to a diameter at a place where the diameter of the cross-sectional shape of the first sensor electrode is long. At this time, the first sensor electrode is stuck into a subject without bending, and therefore, the first sensor electrode can be made hard to break. Further, the major axis of the first sensor electrode is 0.3 mm or less. At this time, the stimulus given by the first sensor electrode to a pain spot is small, and therefore, the subject can be made to feel less pain. Accordingly, the first sensor electrode of the biological information measuring device can be an electrode which is hard to break and gives less pain to a subject.

### Application Example 3

In the biological information measuring device according to the application example, it is preferred that the straight-line distance between the first sensor electrode and the second sensor electrode is 1 mm or more and 50 mm or less.

According to this application example, the distance between the first sensor electrode and the second sensor electrode is 1 mm or more. When the distance between the first sensor electrode and the second sensor electrode is less than 1 mm, the pain felt by a body increases. When the distance between the first sensor electrode and the second sensor electrode is longer than 50 mm, the resistance increases, and therefore, the error in the measurement increases. By setting the distance between the first sensor electrode and the second sensor electrode to 1 mm or more and 50 mm or less, a body can be made to feel less pain, and the biological information can be accurately measured.

### Application Example 4

In the biological information measuring device according to the application example, it is preferred that the first sensor electrode is provided with a sensing detection layer.

According to this application example, the first sensor electrode is provided with a sensing detection layer. Therefore, the sensing detection layer can detect a given biological component and allow an electric current to flow.

### Application Example 5

In the biological information measuring device according to the application example, it is preferred that the in-vivo information is information associated with glucose, the first sensor electrode is a working electrode which is provided with an enzyme layer as the sensing detection layer, and the second sensor electrode is a counter electrode which receives an electric current generated in the first sensor electrode.

According to this application example, the first sensor electrode is a working electrode which is provided with an enzyme layer as the sensing detection layer. Glucose reacts with the enzyme layer to generate an electric current. Then, the second sensor electrode is a counter electrode which receives the electric current generated in the first sensor electrode. As the glucose level is higher, the reaction proceeds further, and therefore, the amount of an electric current increases. Accordingly, by analyzing the amount of an electric current detected by the electric current detection section, a glucose level can be detected.

### Application Example 6

In the biological information measuring device according to the application example, it is preferred that the biological information measuring device further includes a reference electrode which is used for detecting a resistance to an electric current flowing through the first sensor electrode, and the straight-line distance between the first sensor electrode and the reference electrode is shorter than the straight-line distance between the first sensor electrode and the second sensor electrode.

According to this application example, the biological information measuring device further includes a reference electrode which is used for detecting a resistance to an electric current flowing through the first sensor electrode. In a body, interstitial fluid is present. When a substance contained in the interstitial fluid is adhered to the surfaces of the first sensor electrode and the second sensor electrode to form a film thereon, the resistance to an electric current flowing through the first sensor electrode and the second sensor electrode increases. By detecting the resistance using the reference electrode, the electric current detection section can accurately measure the value of an electric current flowing due to glucose. Then, the distance between the first sensor electrode and the reference electrode is set shorter than the distance between the first sensor electrode and the second sensor electrode.

When the resistance to an electric current flowing through the first sensor electrode and the second sensor electrode increases, the electric current detection section increases the voltage to be applied to the second sensor electrode. Therefore, the effect is small even when the distance between the first sensor electrode and the second sensor electrode is far. On the other hand, as the distance between the first sensor electrode and the reference electrode is nearer, the electrodes are less affected by interstitial fluid, and therefore, the increase in the resistance due to the film formed on the electrodes can be accurately detected. In this application example, the distance between the first sensor electrode and the reference electrode is set shorter than the distance between the first sensor electrode and the second sensor electrode. Accordingly, the resistance due to the film formed on the first sensor electrode can be accurately detected and a glucose level can be accurately measured.

### Application Example 7

In the biological information measuring device according to the application example, it is preferred that the straight-line distance between the first sensor electrode and the reference electrode is 1 mm or more and 50 mm or less.

According to this application example, the distance between the first sensor electrode and the reference electrode is 1 mm or more. When the distance between the first sensor electrode and the reference electrode is less than 1 mm, the pain felt by a body increases. When the distance between the first sensor electrode and the reference electrode is longer than 50 mm, the resistance increases, and therefore, the error in the measurement increases. By setting the distance between the first sensor electrode and the reference electrode to 1 mm or more and 50 mm or less, a body can be made to feel less pain, and a glucose level can be accurately measured.

### Application Example 8

In the biological information measuring device according to the application example, it is preferred that the straight-line distance between the reference electrode and the second sensor electrode is 1 mm or more and 50 mm or less.

According to this application example, the distance between the reference electrode and the second sensor electrode is 1 mm or more. When the distance between the reference electrode and the second sensor electrode is less than 1 mm, the pain felt by a body increases. When the distance between the reference electrode and the second sensor electrode is longer than 50 mm, the size of the biological information measuring device increases. Then, when a body is fitted with the biological information measuring device, the body is hard to move, and the inconvenience increases. By setting the distance between the reference electrode and the second sensor electrode to 1 mm or more and 50 mm or less, a glucose level can be measured by the biological information measuring device which gives less pain to a body and has a fitting sense of less inconvenience.

### Application Example 9

In the biological information measuring device according to the application example, it is preferred that the counter electrode contains platinum.

According to this application example, the counter electrode contains platinum. Platinum is highly stable and is less likely to react with interstitial fluid or oxygen, and therefore hardly deteriorates. Accordingly, the counter electrode can stably function for a long period of time.

### Application Example 10

In the biological information measuring device according to the application example, it is preferred that a resin film is placed on the working electrode and the counter electrode.

According to this application example, a resin film is placed on the working electrode and the counter electrode. To the resin film, protein is less likely to adhere. Due to this, even when protein is contained in interstitial fluid, the protein can be prevented from adhering to the surfaces of the working electrode and the counter electrode, so that an electric current can be prevented from becoming difficult to flow.

### Application Example 11

A drug solution supply device according to this application example includes a working electrode, a counter electrode, a reference electrode, a biological information measuring section which measures in-vivo information by applying a predetermined voltage to the reference electrode and detecting an electric current between the working electrode and the counter electrode, a first electrode needle which is inserted into a body by piercing the skin surface, a second electrode needle which is inserted into the body by piercing the skin surface, and an injection needle which is inserted into the body by piercing the skin surface and injects a drug solution into the body, wherein on the circumferential wall surface of each needle of the first electrode needle, the second electrode needle, and the injection needle, any one of the working electrode, the counter electrode, and the reference electrode is placed, and the working electrode, the counter electrode, and the reference electrode are placed on any of the first electrode needle, the second electrode needle, and the injection needle.

According to this application example, the drug solution supply device includes an injection needle, and the injection needle supplies a drug solution to a body. Other than the injection needle, the drug solution supply device includes a working electrode, a counter electrode, a reference electrode, and a biological information measuring section. The biological information measuring section measures in-vivo information by applying a predetermined voltage to the reference electrode and detecting an electric current between the working electrode and the counter electrode.

The working electrode, the counter electrode, and the reference electrode are placed on any of the first electrode needle, the second electrode needle, and the injection needle. Therefore, the device has three needles to be inserted into a body, and the three electrodes are placed on the three needles, and each needle is provided with one electrode. Accordingly, the major axis of each needle can be made short, and thus, a pain spot of a body can be made less stimulated. Further, when the electrode is not placed on the injection needle, three needles are required for placing the electrodes other than the injection needle. Therefore, four needles are required. Since the number of needles in this application example is less than in this case, a pain spot of a body can be made less stimulated. As a result, the drug solution supply device according to this application example enables a body to feel less pain.

### Application Example 12

In the drug solution supply device according to the application example, it is preferred that the in-vivo information is information associated with glucose, and the drug solution is insulin.

According to this application example, the drug solution supply device supplies insulin to a biological tissue. Then, the drug solution supply device detects a glucose level. Therefore, the drug solution supply device can supply insulin according to the glucose level in the body.

### BRIEF DESCRIPTION OF THE DRAWINGS

The invention will be described with reference to the accompanying drawings, wherein like numbers reference like elements.

FIG. 1 is a schematic perspective view showing the structure of a measuring device according to a first embodiment.

FIG. 2 is a schematic perspective view showing the structure of the measuring device.

FIG. 3 is a schematic side cross-sectional view for illustrating a first needle section to a third needle section.

FIG. 4 is a schematic side view showing the structure of the first needle section.

FIG. 5 is a schematic side view of a main part showing the structure of the first needle section.

FIG. 6 is a side cross-sectional view showing the structure of a detection film.

FIG. 7 is a block diagram showing the configuration of a control section.

FIG. 8 is a circuit diagram showing the configuration of an electric current detection circuit.

FIG. 9 is a schematic perspective view showing the structure of a measuring device according to a second embodiment.

FIG. 10 is a circuit diagram showing the configuration of an electric current detection circuit.

FIG. 11 is a schematic perspective view showing the structure of an insulin pump according to a third embodiment.

FIG. 12 is a schematic side view showing the structure of a first needle section.

FIG. 13 is a schematic side view of a main part showing the structure of the first needle section.

FIG. 14 is a block diagram showing the configuration of a control section.

FIG. 15 is a block diagram showing the configuration of a control section according to a fourth embodiment.

FIG. 16 is a block diagram showing the configuration of a control section according to a fifth embodiment.

FIG. 17 is a schematic perspective view showing the structure of an insulin pump according to a sixth embodiment.

FIG. 18 is a schematic side cross-sectional view showing the structures of a first needle section to a third needle section.

### DESCRIPTION OF EXEMPLARY EMBODIMENTS

Hereinafter, embodiments will be described with reference to the accompanying drawings.

Incidentally, the respective members in the respective drawings are shown by changing the scale for each member so as to have a recognizable size in the respective drawings.

### First Embodiment

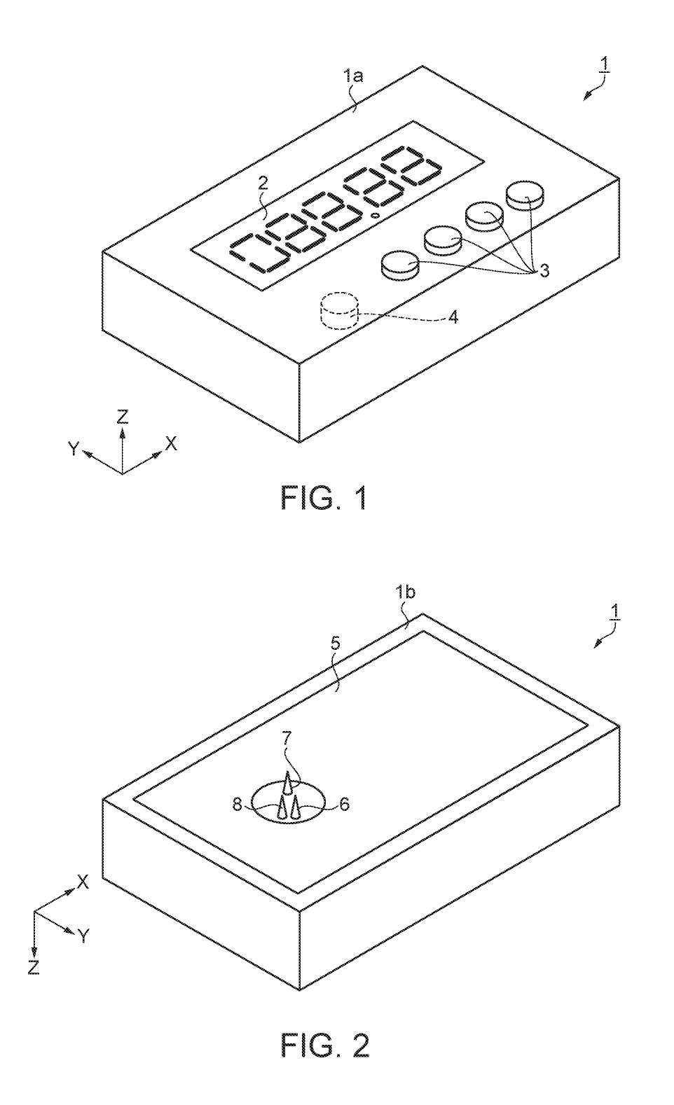

In this embodiment, a characteristic example of a measuring device which measures a glucose level will be described with reference to the accompanying drawings. The measuring device according to the first embodiment will be described with reference to FIGS. 1 to 8. FIGS. 1 and 2 are schematic perspective views showing the structure of the measuring device. FIG. 1 is a view seen from the front side of a measuring device 1 as a biological information measuring device, and FIG. 2 is a view seen from the rear side of the measuring device 1.

As shown in FIG. 1, the measuring device 1 has a planar shape of a rectangular plate. The longitudinal direction of the measuring device 1 is referred to as “X direction”, and the thickness direction of the measuring device 1 is referred to as “Z direction”. The direction orthogonal to the X direction and the Z direction is referred to as “Y direction”. The face on the +Z direction side of the measuring device 1 is referred to as “face 1a”. On the face 1a, a display section 2 and an input section 3 are placed. The display section 2 displays a measured glucose level. As the display section 2, a display device such as an LCD (Liquid Crystal Display) or an OLED (Organic Light-Emitting Diode) is used.

The input section 3 is constituted by a push switch, a rotary knob, or the like. An operator operates the input section 3 and inputs an instruction to start measurement for the measuring device 1 or set the measurement interval or the like. The measuring device 1 further includes a speaker 4. The speaker 4 gives a warning when the measured glucose level is higher than a determination value.

As shown in FIG. 2, the face on the −Z direction side of the measuring device 1 is referred to as “rear face 1b”. On the rear face 1b, an adhesive sheet 5 is placed. The adhesive sheet 5 is used when the measuring device 1 is adhered to the skin of a body. The measuring device 1 is adhered to the skin of a subject by the adhesive sheet 5. On the rear face 1b, on the −X direction side, a first needle section 6 as a first sensor electrode and a working electrode, a second needle section 7 as a second sensor electrode and a counter electrode, and a third needle section 8 as a reference electrode are placed. The first needle section 6 to the third needle section 8 are used by being stuck into the skin of a subject. The first needle section 6 to the third needle section 8 are disposed at the vertices of an isosceles triangle. The distance between the first needle section 6 and the third needle section 8 is shorter than the distance between the first needle section 6 and the second needle section 7, and also shorter than the distance between the second needle section 7 and the third needle section 8.

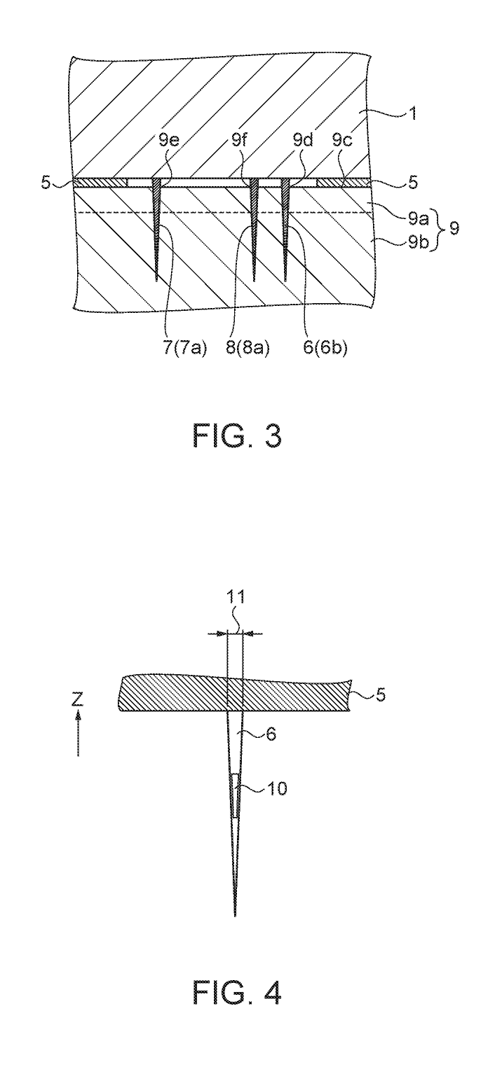

FIG. 3 is a schematic side cross-sectional view for illustrating the first needle section to the third needle section. As shown in FIG. 3, on the surface of a subject 9 as a body, a skin tissue 9a is disposed, and in the inside of the skin tissue 9a, a hypodermal tissue 9b is disposed. The thickness of the skin tissue 9a is about 2 mm. The length of each of the first needle section 6 to the third needle section 8 is about 5 mm. The first needle section 6 to the third needle section 8 are each a needle having a conical shape and each have a sharp tip end. The first needle section 6 to the third needle section 8 are inserted into the subject 9 by piercing the skin surface 9c. The first needle section 6 is inserted into a first insertion place 9d, and the second needle section 7 is inserted into a second insertion place 9e. Further, the third needle section 8 is inserted into a third insertion place 9f. Then, the first needle section 6 to the third needle section 8 penetrate the skin tissue 9a to reach the hypodermal tissue 9b. The hypodermal tissue 9b is filled with interstitial fluid.

On the surface of the first needle section 6, a detection film is placed, and the detection film functions as a working electrode which reacts with interstitial fluid. The second needle section 7 functions as a counter electrode, and the third needle section 8 functions as a reference electrode. The second needle section 7 and the third needle section 8 have electrical conductivity. The first needle section 6 to the third needle section 8 function as electrodes which detect an electric current flowing through interstitial fluid. The electric current generated by the reaction is detected by the first needle section 6 and the second needle section 7. When a film of protein or the like is formed on the surfaces of the needle sections, the film becomes an electrical resistance. The third needle section 8 is an electrode for detecting an increase in the electrical resistance due to the film.

The material of the first needle section 6 is not particularly limited as long as it has a mechanical strength. A material which does not have an influence on a human body is preferred. When platinum is used as the material of the first needle section 6, in the case where adhesion of protein onto platinum of the first needle section 6 is prevented or if there is a concern about metal allergy in a human body, a resin film 6b may be coated on the platinum. As the type of the resin film 6b, although not particularly limited, a film in which a crosslinked structure is constructed by forming a urethane bond using a crosslinking agent such as an isocyanate compound and polymers with a terminal hydroxy group such as polyethylene glycol and 4-hydroxybutyl acrylate (either alone or in admixture) can be used. Then, the first needle section 6 is coated with the constructed resin film 6b. Further, a urea resin may be formed using isocyanate and an amino group as a crosslinking mechanism. In addition, aminopropylpolysiloxane or the like may also be used as the resin film 6b.

Still further, as the resin film 6b, a siloxane resin is preferred, and polydimethylsiloxane is particularly preferred. Further, in the resin film 6b, a mixture obtained by further adding methyl cellulose, acetyl cellulose (cellulose acetate), polyvinylpyrrolidone, polyvinyl alcohol, a polyvinyl alcohol-polyvinyl acetate copolymer, hydroxyethyl methacrylate, and/or poly(2-hydroxyethyl methacrylate), or the like may be used. In order to insolubilize the resin film 6b, isocyanate may be used as a functional group. In the resin film 6b, as a material utilizing UV curability, a poly(vinyl alcohol)-styrylpyridinium compound or the like may be used.

The surface area of the second needle section 7 is preferably large and is preferably one time to two times as large as that of the detection film 10 as the working electrode. The “one time” refers to that the surface area of the second needle section 7 is equal to that of the detection film 10. According to this, the measuring device 1 can highly accurately and stably perform measurement. There is no problem in terms of operation even if the surface area of the second needle section 7 is about half the area of the detection film 10, and therefore, the surface area of the second needle section 7 may be half the area of the detection film 10 as long as there is no problem in terms of the measurement accuracy of the device.

As the constituent material of the second needle section 7, for example, a metal material such as gold, silver, platinum, an alloy containing these metals, a metal oxide-based material such as ITO (Indium Tin Oxide), a carbon-based material such as carbon graphite, a resin, or the like can be used. In the case where an electrically non-conductive material is used, it is used by placing an electrically conductive film on the surface thereof.

In this embodiment, for example, platinum is used as the constituent material of the first needle section 6 and the second needle section 7. Platinum is highly stable and is less likely to react with interstitial fluid or oxygen, and therefore hardly deteriorates. Accordingly, the first needle section 6 and the second needle section 7 can stably function for a long period of time. Further, platinum is a metal which is easy to process, and therefore, the first needle section 6 and the second needle section 7 can be produced with high productivity. As the first needle section 6 and the second needle section 7, a platinum wire containing platinum as a main component may be used. Other than these, any material can be used without any limitation as long as it is a material which is stable without chemically reacting with water, body fluid, blood, or interstitial fluid. As the material of the second needle section 7, a material made of an inorganic material such as glass or silicon, amorphous polyarylate, polysulfone, polyethersulfone, polyphenylene sulfide, polyether ether ketone (another name: aromatic polyether ketone), polyimide, polyetherimide, a fluororesin, nylon, a polyamide including amide, a polyester represented by polyethylene terephthalate, or the like can be used.

In the case where adhesion of protein onto platinum of the second needle section 7 is prevented or if there is a concern about metal allergy in a human body, a resin film 7a may be coated on the platinum. As the resin film 7a, the same film as the resin film 6b on the first needle section 6 can be used.

As the constituent material of the third needle section 8, silver is used. Silver is reversibly transformed between the silver form and the silver ion form in interstitial fluid. By utilizing this reaction, silver is suitable for detecting film formation on the surface of the third needle section 8. Then, the third needle section 8 functions as a reference electrode for detection glucose using an enzyme. It is more preferred that the Ag surface is chlorinated to AgCl.

When a material other than silver is used as the material of the third needle section 8, the same material as that of the second needle section 7 can be used. In the case where adhesion of protein onto platinum of the third needle section 8 is prevented or if there is a concern about metal allergy in a human body, a resin film 8a may be coated on the platinum. As the resin film 8a, the same film as the resin film 6b on the first needle section 6 can be used.

To the resin film 6b, the resin film 7a, and the resin film 8a, protein is less likely to adhere. Due to this, even when protein is contained in blood or interstitial fluid, the protein can be prevented from adhering to the surfaces of the first needle section 6, the second needle section 7, and the third needle section 8, so that an electric current can be prevented from becoming difficult to flow. Further, the resin film 6b prevents the elution of metal ions from the first needle section 6, and the resin film 7a prevents the elution of metal ions from the second needle section 7. In addition, the resin film 8a prevents the elution of metal ions from the third needle section 8. Accordingly, the resin film 6b, the resin film 7a, and the resin film 8a can reduce symptoms of metal allergy even if the subject 9 is susceptible to metal allergy.

FIG. 4 is a schematic side view showing the structure of the first needle section. As shown in FIG. 4, the first needle section 6 has a conical shape, and the detection film 10 is placed on the first needle section 6 from the center to the tip end side. The area of the detection film 10 may be 0.5 mm² or more. The major axis 11 of the first needle section is 0.1 mm or more. The major axis refers to a diameter at a place where the diameter of the cross-sectional shape of the first needle section is long. At this time, the first needle section 6 can be made hard to break. Further, the major axis 11 of the first needle section is 0.3 mm or less. At this time, the stimulus given by the first needle section 6 to a pain spot is small, and therefore, the subject 9 can be made to feel less pain. Accordingly, the first needle section 6 of the measuring device 1 can be an electrode which is hard to break and gives less pain to the subject 9. Further, the major axis 11 of the first needle section is preferably 0.15 mm or more and 0.25 mm or less. According to this, the first needle section 6 can be an electrode which is harder to break and gives less pain to a subject 9.

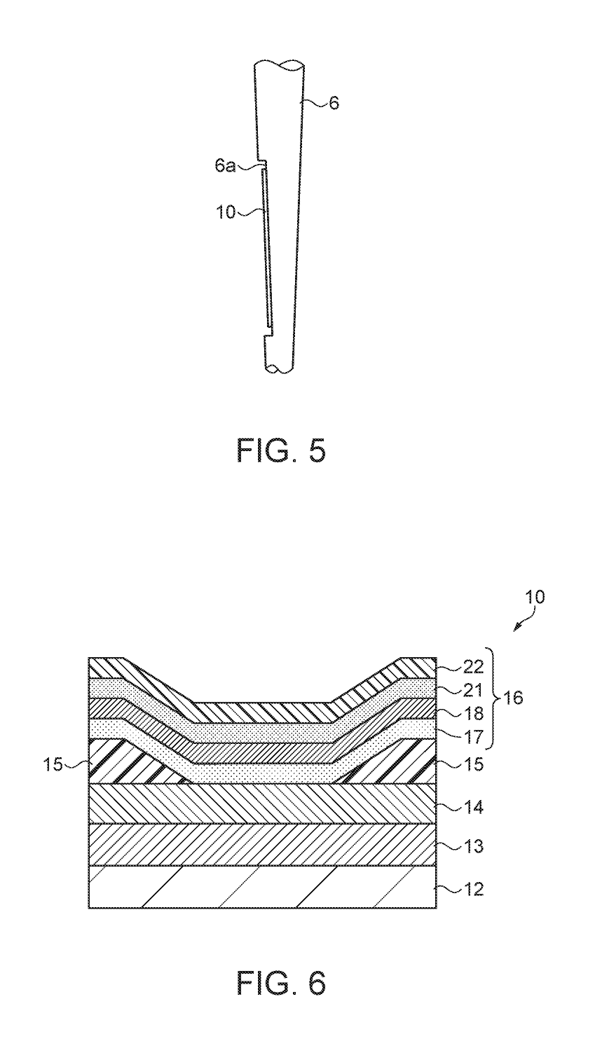

FIG. 5 is a schematic side view of a main part showing the structure of the first needle section. As shown in FIG. 5, a flat face 6a which is a portion of the side surface of the first needle section 6 formed into a flat shape is placed. On this flat face 6a, the detection film 10 is placed. When the detection film 10 is formed using a sputtering method or a vapor deposition method, the detection film 10 is formed on the flat face 6a, and therefore can be formed with a stable film thickness. Accordingly, the detection film 10 having high reliability can be formed.

The measuring device 1 continuously performs detection of glucose in interstitial fluid. Then, the measuring device 1 can be used in a CGMS (continuous glucose monitoring system) which continuously performs observation of a glucose level in interstitial fluid.

Next, a method for detecting glucose by the detection film 10 will be described. In the detection film 10, an enzyme such as glucose oxidase is contained. Glucose is subjected to an enzymatic reaction shown in the formula (1) by an enzyme.

glucose+O₂+H₂O→gluconic acid+H₂O₂ Formula (1)

By the enzymatic reaction, hydrogen peroxide is generated. A voltage is applied to hydrogen peroxide to electrolyze hydrogen peroxide. The voltage is not particularly limited, and may be determined by performing an experiment. In this embodiment, for example, a voltage of 0.6 V is applied.

By the electrolysis, in the first needle section 6, a reaction represented by the formula (2) occurs. In the second needle section 7, a reaction represented by the formula (3) occurs.

H₂O₂→O₂+2H⁺+2e⁻ Formula (2)

2H⁺+1/2O₂+2e⁻→H₂O Formula (3)

As shown in the formula (2), in the first needle section 6, oxygen, hydrogen ions, and electrons are generated. Then, as shown in the formula (3), in the second needle section 7, electrons, oxygen, and hydrogen ions supplied from the first needle section 6 react with one another, whereby the concentration of hydroxide ions increases. In this manner, electrons move between the first needle section 6 and the second needle section 7, and therefore, by measuring the amount of an electric current, the amount of hydrogen peroxide can be quantitatively determined. Then, by detecting the amount of generated hydrogen peroxide, a glucose level is calculated.

FIG. 6 is a side cross-sectional view showing the structure of a detection film. Next, the structure of the detection film 10 will be described. As shown in FIG. 6, the detection film 10 includes a base layer 12. On the base layer 12, an ITO (Indium Tin Oxide) electrode layer 13 and an enzyme electrode layer 14 are placed. On the enzyme electrode layer 14, a partition wall layer 15 is placed along the outer circumference of the enzyme electrode layer 14. The partition wall layer 15 opens on the inside, and the enzyme electrode layer 14 is exposed on the inside.

A sensing layer 16 is placed overlapped on the enzyme electrode layer 14 and the partition wall layer 15. The sensing layer 16 is stacked on the enzyme electrode layer 14 on the inside of the partition wall layer 15. The sensing layer 16 is constituted by four layers. The sensing layer 16 includes a noise removal layer 17, a detection layer 18 as a sensing detection layer and an enzyme layer, a protective layer 21, and a control layer 22, which are stacked in this order from the enzyme electrode layer 14 side.

The base layer 12 is a layer which insulates the first needle section 6 from the ITO electrode layer 13. The base layer 12 is not particularly limited as long as it is an insulating material which is stable in the air without chemically reacting with water, body fluid, blood, or interstitial fluid. As the base layer 12, an inorganic material such as glass or silicon can be used as a starting material. Other than these, amorphous polyarylate, polysulfone, polyethersulfone, or polyphenylene sulfide can be used in the base layer 12. Other than these, polyether ether ketone (another name: aromatic polyether ketone) or polyimide can be used in the base layer 12. Other than these, polyetherimide, a fluororesin, nylon, a polyamide including amide, a polyester represented by polyethylene terephthalate, or the like can be used in the base layer 12.

The ITO electrode layer 13 is an electrically conductive layer to serve as a wire. The ITO electrode layer 13 is a layer which electrically connects the detection film 10 and the control section. In the detection film 10, the enzyme electrode layer 14 is placed overlapped on the ITO electrode layer 13. The ITO electrode layer 13 may be coated with an insulating film at a place other than the detection film 10. Accordingly, application of noise to an electric current flowing through the ITO electrode layer 13 can be suppressed.

The enzyme electrode layer 14 is not particularly limited as long as it can be used as an enzyme electrode. In the enzyme electrode layer 14, platinum, gold, an alloy of these metals, an alloy containing these metals as main materials, carbon graphite, or the like can be used. In this embodiment, for example, as the material of the enzyme electrode layer 14, platinum is used. In the case where the enzyme electrode layer 14 is platinum, gold, or an alloy of these metals, the enzyme electrode layer 14 is formed by a sputtering method, a plating method, or a vacuum heating vapor deposition method. In the case where the enzyme electrode layer 14 is carbon graphite, carbon graphite is mixed in a binder having been dissolved in an appropriate solvent, and the resulting mixture is applied, whereby the enzyme electrode layer 14 is formed. In order to accurately control the electrode area of the enzyme electrode layer 14, it is preferred to use platinum as the material of the enzyme electrode layer 14. The film is formed using a sputtering method or a plating method, and then patterned using a photolithographic technique. By doing this, the enzyme electrode layer 14 can be accurately placed.

The partition wall layer 15 is placed for improving the adhesiveness between the enzyme electrode layer 14 and the sensing layer 16. The sensing layer 16 has hydrophilicity, and is swollen by absorbing interstitial fluid. The swollen sensing layer 16 is easy to peel off from the enzyme electrode layer 14. The partition wall layer 15 is not particularly limited as long as it is a material from which the swollen sensing layer 16 is difficult to peel off. As the material of the partition wall layer 15, for example, polyimide or an acrylic material can be used. Then, the partition wall layer 15 is formed by a photolithographic method or a photoresist method using a dry resist sheet.

The noise removal layer 17 prevents a compound such as acetaminophen, ascorbic acid, or uric acid from permeating the detection film 10 to reach the enzyme electrode layer 14. A compound such as acetaminophen, ascorbic acid, or uric acid may be contained in interstitial fluid. In order to realize this function, as the material of the noise removal layer 17, although not particularly limited, methyl cellulose, acetyl cellulose (cellulose acetate), polyvinylpyrrolidone, polyvinyl alcohol, or the like can be used. Other than these, a polyvinyl alcohol-polyvinyl acetate copolymer, hydroxyethyl methacrylate or poly(2-hydroxyethyl methacrylate) can be used as the material of the noise removal layer 17. These materials may be used in combination. In order to insolubilize the noise removal layer 17, isocyanate may be used as a functional group. As a material utilizing UV curability, a poly(vinyl alcohol)-styrylpyridinium compound or the like can also be used. Further, albumin may be contained for the purpose of protecting the lower boundary surface of the detection layer 18.

The detection layer 18 is a layer containing an enzyme. The detection layer 18 contains an enzyme, a resin containing an enzyme, a binder or a curing agent, and albumin which protects and stabilizes the enzyme. As the enzyme, glucose oxidase is contained, and the glucose oxidase promotes the enzymatic reaction represented by the formula (1).

As the resin material of the resin containing the enzyme of the detection layer 18, although not particularly limited, for example, methyl cellulose, acetyl cellulose (cellulose acetate), polyvinylpyrrolidone, polyvinyl alcohol, a polyvinyl alcohol-polyvinyl acetate copolymer, or the like is preferred. Among these, one type or two or more types in combination may be used. By using these resin materials, the decrease in the activity of the enzyme can be accurately suppressed. The resin material is not limited thereto, and may be any as long as the material does not significantly decrease the activity of the enzyme.

As the binder and the curing agent of the detection layer 18, a material using a material having two or more functional groups such as aldehyde or isocyanate in the molecule, a polymer material having a functional group which can be bonded to such a functional group, specifically, a hydroxy group, an amino group, an epoxy group, or the like at a terminal, and an enzyme are mixed, and the resulting material can be used. As a specific example of the binder and the curing agent, glutaraldehyde, toluene diisocyanate, isophorone diisocyanate, or the like can be used. Further, as the material utilizing UV curability, a poly(vinyl alcohol)-styrylpyridinium compound or the like can also be used.

As the albumin, human albumin or bovine albumin can be used. In albumin, a phosphate buffer component may be contained. By doing this, the change in pH due to the enzymatic reaction can be suppressed.

The protective layer 21 protects the upper boundary surface of the detection layer 18. In order to realize this function, as the material of the protective layer 21, although not particularly limited, methyl cellulose, acetyl cellulose (cellulose acetate), polyvinylpyrrolidone, polyvinyl alcohol, a polyvinyl alcohol-polyvinyl acetate copolymer, and the like can be used alone or in combination. Further, it is preferred to contain albumin in the material of the protective layer 21.

In order to insolubilize the protective layer 21, in the material of the protective layer 21, a binder or a curing agent may be contained. As the binder or the curing agent, a material using a material having two or more functional groups such as aldehyde or isocyanate in the molecule, a polymer material having a functional group which can be bonded to such a functional group, specifically, a hydroxy group, an amino group, an epoxy group, or the like at a terminal, and an enzyme are mixed, and the resulting material can be used. As a specific example of the binder or the curing agent of the protective layer 21, glutaraldehyde, toluene diisocyanate, isophorone diisocyanate, or the like can be used. Further, as the material utilizing UV curability, a poly(vinyl alcohol)-styrylpyridinium compound or the like can also be used. As the albumin, human albumin or bovine albumin can be used.

The control layer 22 prevents blood, interstitial fluid, or the like which is the measurement object from being direct contact with the detection layer 18. The control layer 22 has a function to control the permeability of oxygen and glucose by allowing oxygen and glucose to permeate partially. It is preferred that the control layer 22 allows oxygen to permeate more than glucose.

When glucose reaches the detection layer 18 at a glucose level which exceeds the detectable level of the detection layer 18, the enzymatic reaction reaches a saturated state. At this time, the glucose level cannot be detected. The control layer 22 controls the permeability of glucose, and even when the glucose level in the measurement object is high, the enzymatic reaction is prevented from reaching a saturated state.

The material of the control layer 22 is not particularly limited as long as it can control the permeability of oxygen and glucose. As the material of the control layer 22, a material in which a crosslinked structure is constructed by forming a urethane bond using a crosslinking agent such as an isocyanate compound and polymers with a terminal hydroxy group such as polyethylene glycol and 4-hydroxybutyl acrylate (either alone or in admixture) can be used. Other than this, aminopropylpolysiloxane or the like obtained by forming a urea resin using isocyanate and an amino group can be used as the material of the control layer 22. In addition thereto, as the material of the control layer 22, a siloxane resin is preferred, and polydimethylsiloxane is particularly preferred.

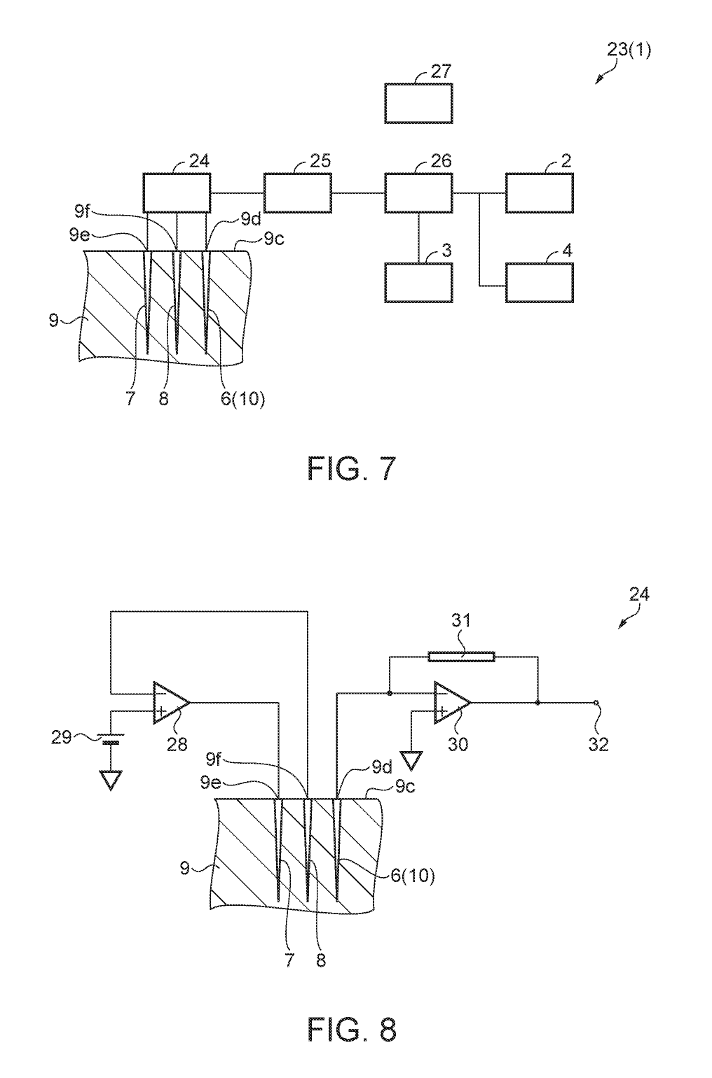

FIG. 7 is a block diagram showing the configuration of the control section. The control section 23 shown in FIG. 7 is placed in the inside of the measuring device 1. The control section 23 includes an electric current detection circuit 24 as the electric current detection section, and to the electric current detection circuit 24, the first needle section 6 to the third needle section 8 are connected. The electric current detection circuit 24 is further connected to an A/D conversion circuit 25 (Analog/Digital). The electric current detection circuit 24 detects an electric current flowing between the detection film 10 and the second needle section 7. Then, the electric current is converted to a voltage and output to the A/D conversion circuit 25. The electric current flowing between the detection film 10 and the second needle section 7 correlates with a glucose level, and therefore, the voltage output to the A/D conversion circuit 25 has a correlation with a glucose level.

The A/D conversion circuit 25 converts the voltage to a numerical value. The A/D conversion circuit 25 is connected to a central processing section 26 and outputs the data of voltage converted to the numerical value to the central processing section 26. In the central processing section 26, a display section 2, an input section 3, and a speaker 4 are placed. The central processing section 26 converts the input data of voltage to a glucose level. The central processing section 26 stores the data of a correlation table showing the relationship between the data of voltage and the glucose level. Then, the central processing section 26 calculates the glucose level using the correlation table and the data of voltage.

The central processing section 26 outputs the calculated glucose level to the display section 2, and the display section 2 displays the glucose level. Further, the central processing section 26 stores the data of the determination value, and then, compares the determination value with the glucose level and determines whether the glucose level is within the normal range or is a value in an abnormal state. When the central processing section 26 determines that the glucose level is a value in an abnormal state, the information that the glucose level is a value in an abnormal state is output to the display section 2 and the speaker 4. Then, the display section 2 displays a warning statement, and the speaker 4 emits a warning sound.

The operator operates the input section 3 and inputs an instruction to start measurement, stop measurement, or the like to the central processing section 26. The central processing section 26 drives the measuring device 1 according to the instruction of the operator. In the measuring device 1, an electric battery 27 is placed, and the electric battery 27 supplies an electric power to the control section 23. According to this, the measuring device 1 can be attached to the subject 9 and driven without being connected to the external power supply. Accordingly, the subject 9 can easily move while being fitted with the measuring device 1.

FIG. 8 is a circuit diagram showing the configuration of the electric current detection circuit. The electric current detection circuit 24 shown in FIG. 8 is a circuit called “potentiostat”. The electric current detection circuit 24 includes a first operational amplifier 28. A power supply 29 is connected to the positive input terminal of the first operational amplifier 28, and a predetermined voltage is applied to the positive input terminal of the first operational amplifier 28. The voltage value is not particularly limited, but is set to, for example, 0.6 V in this embodiment. The power supply 29 can be realized by dividing the voltage of the electric battery 27 and setting the voltage in a diode. To the negative input terminal of the first operational amplifier 28, the third needle section 8 is connected, and to the output terminal of the first operational amplifier 28, the second needle section 7 is connected. In the first operational amplifier 28, an electric current is allowed to flow through the second needle section 7 so that the potential from the third needle section 8 and the potential to be applied by the power supply 29 are the same. Then, the first operational amplifier 28 works so that an electric current does not flow through the third needle section 8.

The electric current detection circuit 24 includes a second operational amplifier 30. The positive input terminal of the second operational amplifier 30 is grounded. Then, to the negative input terminal of the second operational amplifier 30, the detection film 10 is connected, and the negative input terminal and the output terminal of the second operational amplifier 30 are connected through a resistance 31. Then, the output terminal 32 of the electric current detection circuit 24 is connected to the output terminal of the second operational amplifier 30.

In the second operational amplifier 30, the positive input terminal and the negative input terminal have the same potential, and therefore, the potential of the detection film 10 is 0 V. Then, between the third needle section 8 and the detection film 10, a potential of 0.6 V is generated. In the subject 9, the enzymatic reactions represented by the formulae (1) to (3) occur, and therefore, an electric current flows between the second needle section 7 and the detection film 10. The second operational amplifier 30 has a high input impedance, and therefore, an electric current flows from the detection film 10 to the output terminal of the second operational amplifier 30 through the resistance 31. When the electric current flows through the resistance 31, a voltage drop occurs, and therefore, at the output terminal 32, the electric current between the second needle section 7 and the detection film 10 is converted to a voltage and output.

A compensation circuit (not shown) is placed in the electric current detection circuit 24, and the compensation circuit detects the resistance between the third needle section 8 and the detection film 10. When a coating film is formed on the surfaces of the second needle section 7, the third needle section 8, and the detection film 10, the resistance increases. The compensation circuit changes the voltage to be applied to the second needle section 7 according to the resistance between the third needle section 8 and the detection film 10. As a result, an electric current can be stably measured for a long period of time by the compensation circuit.

The detection film 10, the second needle section 7, and the third needle section 8 are disposed so that the distance between the detection film 10 and the third needle section 8 is shorter than the distance between the detection film 10 and the second needle section 7. Even when the resistance to the electric current flowing through the detection film 10 and the second needle section 7 increases due to the coating film, the electric current detection circuit 24 performs compensation by allowing an electric current to flow through the second needle section 7 so that the potential from the third needle section 8 and the potential to be applied by the power supply 29 are the same. Accordingly, the effect is small even when the distance between the detection film 10 and the second needle section 7 is far. On the other hand, as the distance between the detection film 10 and the third needle section 8 is nearer, these members are less affected by interstitial fluid, and therefore, the increase in the resistance due to the coating film formed on the detection film 10 can be accurately detected. In this embodiment, the distance between the detection film 10 and the third needle section 8 is set shorter than the distance between the detection film 10 and the second needle section 7. Accordingly, the resistance due to the coating film formed on the detection film 10 can be accurately detected and a glucose level can be accurately measured.

The distance between the first needle section 6 and the third needle section 8 is set to 1 mm or more and 50 mm or less. When the distance between the first needle section 6 and the third needle section 8 is less than 1 mm, the pain felt by the subject 9 increases. When the distance between the detection film 10 of the first needle section 6 and the third needle section 8 is longer than 50 mm, the resistance to interstitial fluid increases, and therefore, the error in the measurement increases. By setting the distance between the first needle section 6 and the third needle section 8 to 1 mm or more and 50 mm or less, the subject 9 can be made to feel less pain, and the glucose level can be accurately measured.

The distance between the third needle section 8 and the second needle section 7 is set to 1 mm or more and 50 mm or less. When the distance between the third needle section 8 and the second needle section 7 is less than 1 mm, the pain felt by the subject 9 increases. When the distance between the third needle section 8 and the second needle section 7 is longer than 50 mm, the size of the measuring device 1 increases. Then, when the subject 9 is fitted with the measuring device 1, the subject 9 is hard to move, and the inconvenience increases. By setting the distance between the third needle section 8 and the second needle section 7 to 1 mm or more and 50 mm or less, a glucose level can be accurately measured by the measuring device 1 which gives less pain to the subject 9 and has a fitting sense of less inconvenience.

As described above, according to this embodiment, the following effects are obtained.

(1) According to this embodiment, the measuring device 1 includes the first needle section 6, the second needle section 7, and the electric current detection circuit 24. On the surface of the first needle section 6, the detection film 10 is placed, and the detection film 10 functions as a working electrode. The electrically conductive second needle section 7 functions as a counter electrode. When interstitial fluid of the subject 9 comes in contact with the detection film 10, glucose in the interstitial fluid reacts with the detection film 10, and electrons are generated. Then, electrons move between the detection film 10 and the second needle section 7 to generate an electric current. The electric current detection circuit 24 detects the amount of the electric current. As the glucose level is higher, the amount of the generated electrons increases, and therefore, by detecting the amount of the electric current by the electric current detection circuit 24, the glucose level in the interstitial fluid can be detected.

(2) According to this embodiment, the detection film 10 is placed on the surface of the first needle section 6. In the case of the related art, on a substrate, a detection film 10, a counter electrode, and a reference electrode are placed, and the substrate is disposed in the inside of a needle having a cylindrical shape. Due to this, as compared with the needle in the case of the related art, the major axis of the first needle section 6 can be made shorter. Since the electrically conductive second needle section 7 functions as a counter electrode, the major axis of the second needle section 7 can be made shorter than that of the needle having a cylindrical shape in the case of the related art. As the major axes of the first needle section 6 and the second needle section 7 are shorter, a pain spot of the subject 9 is less stimulated. Therefore, the measuring device 1 of this embodiment enables the subject 9 to feel less pain even when the first needle section 6 to the third needle section 8 are stuck into the subject 9.

(3) According to this embodiment, the major axis 11 of the first needle section is 0.1 mm or more. At this time, the first needle section 6 is stuck into the subject 9 without bending, and therefore, the first needle section 6 can be made hard to break. Further, the major axis 11 of the first needle section is 0.3 mm or less. At this time, the stimulus given by the first needle section 6 to a pain spot is small, and therefore, the subject 9 can be made to feel less pain. Accordingly, the first needle section 6 of the measuring device 1 can be an electrode which is hard to break and gives less pain to the subject 9.

(4) According to this embodiment, the second needle section 7 which functions as a counter electrode contains platinum. Platinum is highly stable and is less likely to react with interstitial fluid or oxygen, and therefore hardly deteriorates. Accordingly, the second needle section 7 can stably function for a long period of time.

(5) According to this embodiment, on the surface of the first needle section 6, the resin film 6b is placed, on the surface of the second needle section 7, the resin film 7a is placed, and on the surface of the third needle section 8, the resin film 8a is placed. To the resin film 6b, the resin film 7a, and the resin film 8a, protein is less likely to adhere. Due to this, even when protein is contained in interstitial fluid, the protein can be prevented from adhering to the surfaces of the first needle section 6 to the third needle section 8, so that an electric current can be prevented from becoming difficult to flow. Further, the resin film 6b, the resin film 7a, and the resin film 8a prevent the elution of a metal contained in each needle in interstitial fluid. Accordingly, symptoms can be reduced even if the subject 9 is susceptible to metal allergy.

(6) According to this embodiment, the measuring device 1 includes the third needle section 8 which is used for detecting the resistance to an electric current flowing through the detection film 10. When a substance contained in interstitial fluid is adhered to the surfaces of the detection film 10 and the second needle section 7 to form a film thereon, the resistance to an electric current flowing through the detection film 10 and the second needle section 7 increases. By detecting the resistance using the third needle section 8, the electric current detection circuit 24 can accurately measure the value of an electric current flowing due to glucose. Then, the distance between the detection film 10 and the third needle section 8 is set shorter than the distance between the detection film 10 and the second needle section 7. When the resistance to an electric current flowing through the detection film 10 and the second needle section 7 increases, the electric current detection circuit 24 increases the voltage to be applied to the second needle section 7. Therefore, the effect is small even when the distance between the detection film 10 and the second needle section 7 is far. On the other hand, as the distance between the detection film 10 and the third needle section 8 is nearer, the members are less affected by interstitial fluid, and therefore, the increase in the resistance due to the film formed on the electrodes can be accurately detected. In this embodiment, the distance between the detection film 10 and the third needle section 8 is set shorter than the distance between the detection film 10 and the second needle section 7. Accordingly, the resistance due to the film formed on the detection film 10 can be accurately detected and a glucose level can be accurately measured.

(7) According to this embodiment, the distance between the first needle section 6 and the third needle section 8 is set to 1 mm or more and 50 mm or less. When the distance between the first needle section 6 and the third needle section 8 is less than 1 mm, the pain felt by the subject 9 increases. When the distance between the detection film 10 of the first needle section 6 and the third needle section 8 is longer than 50 mm, the resistance to interstitial fluid increases, and therefore, the error in the measurement increases. By setting the distance between the first needle section 6 and the third needle section 8 to 1 mm or more and 50 mm or less, the subject 9 can be made to feel less pain, and the glucose level can be accurately measured.

(8) According to this embodiment, the distance between the third needle section 8 and the second needle section 7 is set to 1 mm or more and 50 mm or less. When the distance between the third needle section 8 and the second needle section 7 is less than 1 mm, the pain felt by the subject 9 increases. When the distance between the third needle section 8 and the second needle section 7 is longer than 50 mm, the size of the measuring device 1 increases. Then, when the subject 9 is fitted with the measuring device 1, the subject 9 is hard to move, and the inconvenience increases. By setting the distance between the third needle section 8 and the second needle section 7 to 1 mm or more and 50 mm or less, a glucose level can be measured by the measuring device 1 which gives less pain to the subject 9 and has a fitting sense of less inconvenience.

### Second Embodiment

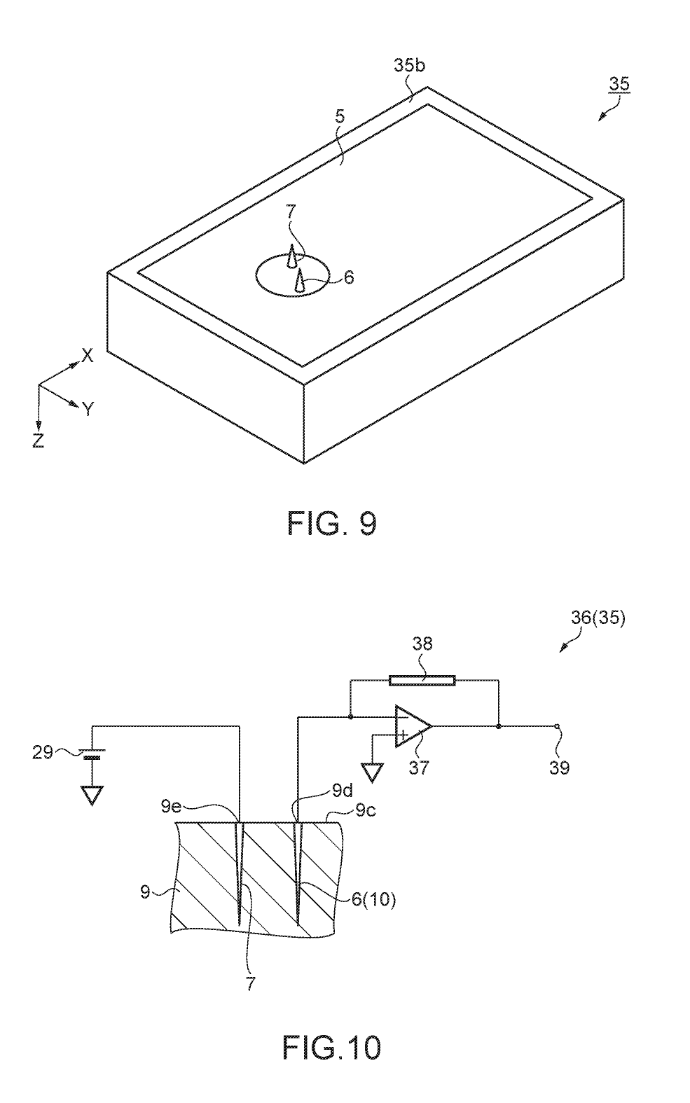

Next, one embodiment of a measuring device will be described with reference to FIGS. 9 and 10. FIG. 9 is a schematic perspective view showing the structure of a measuring device. FIG. 10 is a circuit diagram showing the configuration of an electric current detection circuit. This embodiment is different from the first embodiment in that a third needle section 8 which functions as a reference electrode is omitted. Incidentally, the description of the same points as those of the first embodiment will be omitted.

That is, in this embodiment, as shown in FIG. 9, a first needle section 6 and a second needle section 7 are placed on the rear face 35b of a measuring device 35. The first needle section 6 and the second needle section 7 are used by being stuck into the skin of a subject.

As shown in FIG. 10, the measuring device 35 includes an electric current detection circuit 36. The electric current detection circuit 36 is a circuit which measures an electric current flowing between the second needle section 7 and a detection film 10. The electric current detection circuit 36 includes an operational amplifier 37. The positive input terminal of the operational amplifier 37 is grounded. Then, to the negative input terminal of the operational amplifier 37, the detection film 10 is connected, and the negative input terminal and the output terminal of the operational amplifier 37 are connected through a resistance 38. Then, the output terminal 39 of the electric current detection circuit 36 is connected to the output terminal of the operational amplifier 37.

The second needle section 7 is connected to a power supply 29. In a subject 9, the enzymatic reactions represented by the formulae (1) to (3) occur, and therefore, an electric current flows between the second needle section 7 and the detection film 10. An electric current flowing between the second needle section 7 and the detection film 10 is input to the output terminal of the operational amplifier 37 through the resistance 38. Then, the electric current input to the output terminal flows into the second needle section 7 from the ground. The voltage of the negative input terminal of the operational amplifier 37 is 0 V, and the voltage of the output terminal 39 becomes a value obtained by multiplying the resistance value of the resistance 38 by the value of the electric current flowing through the resistance 38 due to the voltage drop in the resistance 38.

The impedance of the input terminal of the operational amplifier 37 is high, and therefore, an electric current flowing between the second needle section 7 and the detection film 10 and an electric current flowing through the resistance 38 are the same. Therefore, by measuring the voltage of the output terminal 39, the electric current flowing between the second needle section 7 and the detection film 10 can be measured. The second needle section 7 corresponds to a counter electrode, and the detection film 10 corresponds to a working electrode. Therefore, by measuring the voltage of the output terminal 39, the glucose level in the subject 9 can be measured.

The distance between the first needle section 6 and the second needle section 7 is 1 mm or more and 50 mm or less. When the distance between the first needle section 6 and the second needle section 7 is less than 1 mm, the pain felt by the subject 9 increases. When the distance between the first needle section 6 and the second needle section 7 is longer than 50 mm, the resistance increases, and therefore, the error in the measurement increases. By setting the distance between the first needle section 6 and the second needle section 7 to 1 mm or more and 50 mm or less, the subject 9 can be made to feel less pain, and the glucose level can be accurately measured. Accordingly the measuring device 35 enables the subject 9 to feel less pain and can accurately measure a glucose level.

Also in this embodiment, in the same manner as in the first embodiment, the major axes of the first needle section 6 and the second needle section 7 are short, and therefore, a glucose level can be measured without stimulating a pain spot of the subject 9 even when the first needle section 6 and the second needle section 7 are stuck into the subject 9. Therefore, the measuring device 35 of this embodiment enables the subject 9 to feel less pain even when the first needle section 6 and the second needle section 7 are stuck into the subject 9.

As described above, according to this embodiment, the following effect is obtained.

(1) According to this embodiment, the distance between the first needle section 6 and the second needle section 7 is 1 mm or more and 50 mm or less. As a result, the subject 9 can be made to feel less pain, and the glucose level can be accurately measured.

### Third Embodiment

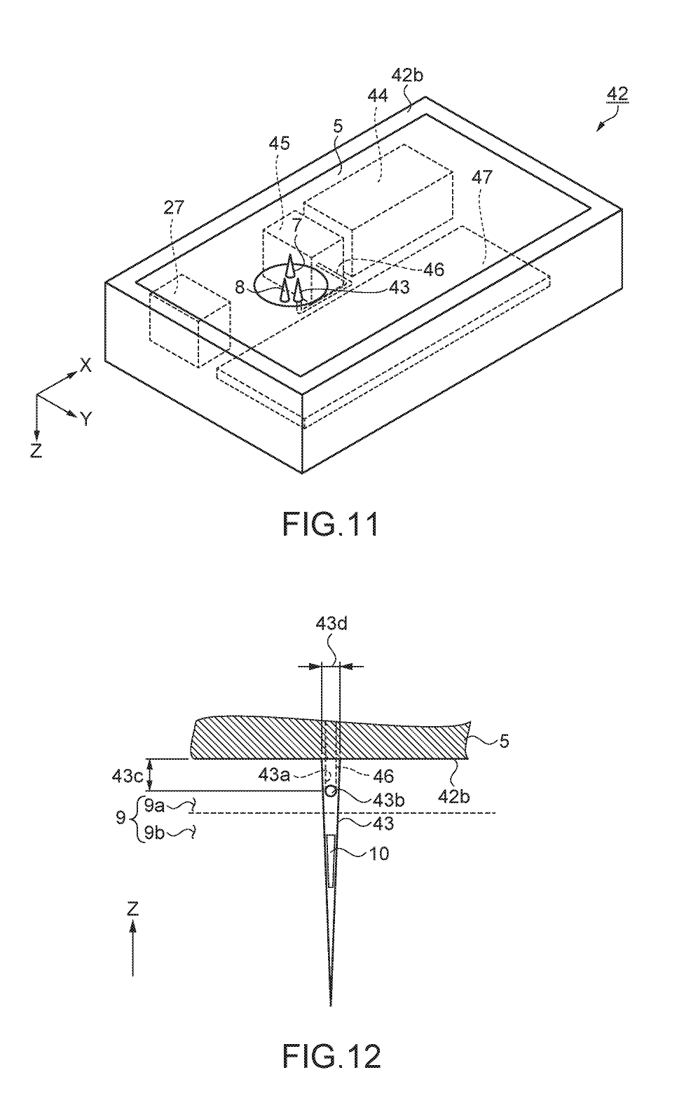

Next, one embodiment of an insulin pump will be described with reference to FIGS. 11 to 14. In the insulin pump of this embodiment, the measuring device 1 described in the first embodiment is used. Incidentally, the description of the same points as those of the first embodiment will be omitted.

FIG. 11 is a schematic perspective view showing the structure of an insulin pump, and is a view seen from the rear side of an insulin pump 42 as a drug solution supply device. That is, in this embodiment, as shown in FIG. 11, on the rear face 42b of the insulin pump 42, a first needle section 43 as an injection needle, a first sensor electrode, and a working electrode, a second needle section 7, and a third needle section 8 are placed. In the inside of the insulin pump 42, a tank 44 which stores insulin as a drug solution is placed. Further, in the inside of the insulin pump 42, a pump 45 which transports insulin stored in the tank 44 is placed. The pump 45 and the first needle section 43 are connected through a pipe 46. Other than these, an electric battery 27 and a circuit substrate 47 are placed in the inside of the insulin pump 42.

In the inside of the tank 44, a bag made of a resin is included, and insulin is stored in the bag. Then, as the insulin in the tank 44 decreases, the bag is deformed. According to this, insulin can be stored without being in contact with air. Further, the pump 45 is preferably a volume pump capable of controlling the transport amount, and a gear pump, a screw pump, a vane pump, or the like can be used. On the circuit substrate 47, a circuit which controls the pump 45 and a circuit which measures a glucose level are placed.

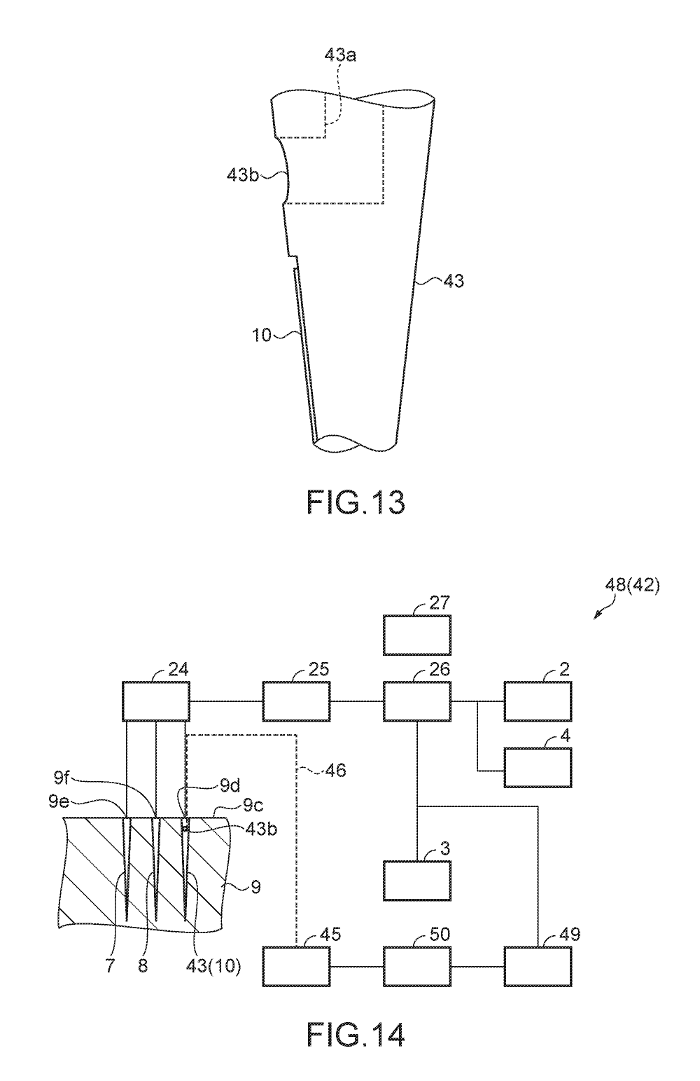

FIG. 12 is a schematic side view showing the structure of the first needle section, and FIG. 13 is a schematic side view of a main part showing the structure of the first needle section. As shown in FIGS. 12 and 13, the first needle section 43 has a conical shape in the same manner as the first needle section 6 of the first embodiment, and the major axis 43d of the first needle section is 0.1 mm or more and 0.3 mm or less. Therefore, the first needle section 43 of the insulin pump 42 can be made hard to break and enables the subject 9 to feel less pain. A detection film 10 is placed on the first needle section 43 from the center to the tip end side. Then, in the inside of the first needle section 43, a hole 43a is placed, and the hole 43a is connected to an opening section 43b as a hole section located between the root of the first needle section 43 and the detection film 10.

When the pump 45 is driven, insulin is discharged from the tank 44, passes through the pump 45, the pipe 46, and the hole 43a, and flows out from the opening section 43b of the first needle section 43 to a subject 9. The distance 43c between the center of the opening section 43b and the rear face 42b is set within the range of 1 mm or more and 2 mm or less. At this time, the opening section 43b is located in a skin tissue 9a, and therefore, insulin can be supplied to the skin tissue 9a. Then, a glucose level in a hypodermal tissue 9b can be measured. Interstitial fluid is present more in the hypodermal tissue 9b than in the skin tissue 9a. There are more chances for glucose to come in contact with the detection film 10 when the detection film 10 is located in a place where interstitial fluid is present more. Therefore, a glucose level can be more accurately measured in the case where the detection film 10 is located in the hypodermal tissue 9b than in the case where the detection film 10 is located in the skin tissue 9a.

FIG. 14 is a block diagram showing the configuration of a control section. A control section 48 as a biological information measuring section shown in FIG. 14 is placed on the circuit substrate 47 located in the inside of the insulin pump 42. The control section 48 includes an electric current detection circuit 24, an A/D conversion circuit 25, a central processing section 26, a display section 2, an input section 3, and a speaker 4 in the same manner as in the first embodiment. Further, the control section 48 includes a supply amount control section 49 which controls the supply amount of insulin and a pump driving section 50 which drives the pump 45. The supply amount control section 49 is connected to the central processing section 26, and further connected to the pump driving section 50. Then, the pump driving section 50 is connected to the pump 45.

The first needle section 43 is inserted into the subject 9 by piercing the skin surface 9c at a first insertion place 9d. The second needle section 7 is inserted into the subject 9 by piercing the skin surface 9c at a second insertion place 9e. The third needle section 8 is inserted into the subject 9 by piercing the skin surface 9c at a third insertion place 9f.

The central processing section 26 calculates the measured glucose level and compares it with the determination value. Then, when the central processing section 26 determines that the glucose level is a value in an abnormal state, the central processing section 26 outputs an instruction signal to discharge insulin and the data of the glucose level to the supply amount control section 49. The supply amount control section 49 calculates the amount of insulin to be supplied to the subject 9. The supply amount control section 49 includes a supply amount table showing the relationship between the glucose level and the amount of insulin to be supplied. Then, the supply amount control section 49 calculates the amount of insulin to be supplied with reference to the glucose level and the supply amount table.

Further, the supply amount control section 49 calculates a time for driving the pump 45 based on the amount of insulin to be supplied. For the pump 45, the amount of insulin to be supplied per unit time has been set. Then, the supply amount control section 49 outputs the data of the time for driving the pump 45 and an instruction signal to drive the pump 45 to the pump driving section 50. The pump driving section 50 drives the pump 45 according to the instruction signal. Then, the pump 45 supplies the instructed amount of insulin to the subject 9.

The first needle section 43 corresponds to an injection needle. One of the second needle section 7 and the third needle section 8 corresponds to a first electrode needle, and the other corresponds to a second electrode needle. Then, on the circumferential wall surface of each needle of the first electrode needle, the second electrode needle, and the injection needle, any one of a working electrode, a counter electrode, and a reference electrode is placed, and the working electrode, the counter electrode, and the reference electrode are placed on any of the first electrode needle, the second electrode needle, and the injection needle.

As described above, according to this embodiment, the following effects are obtained.

(1) According to this embodiment, the insulin pump 42 includes the first needle section 43, and the first needle section 43 supplies insulin to the subject 9 from the opening section 43b. Then, on the first needle section 43, the detection film 10 is placed. Further, the insulin pump 42 includes the second needle section 7 and the electric current detection circuit 24. The first needle section 43 and the second needle section 7 are used by being stuck into the subject 9. Then, the detection film 10 comes in contact with a biological tissue. In the subject 9, interstitial fluid is contained, and in the interstitial fluid, glucose is contained. When the detection film 10 reacts with glucose, an electric current flows between the detection film 10 and the second needle section 7. Then, by detecting the electric current by the electric current detection circuit 24, the glucose level in the interstitial fluid can be detected.

(2) According to this embodiment, the detection film 10 is placed on the first needle section 43. Therefore, a pain spot of the subject 9 can be made less stimulated as compared with the case where a needle of the detection film 10 is inserted into the subject 9 separately from the first needle section 43. As a result, the insulin pump 42 enables the subject 9 to feel less pain.

(3) According to this embodiment, in the first needle section 43, the opening section 43b and the detection film 10 are placed. Then, the opening section 43b is located closer to the root of the first needle section 43 than the detection film 10. Due to this, the detection film 10 is located in a deeper place than the opening section 43b. Accordingly, the detection film 10 can be located in a place where interstitial fluid is present more. As a result, glucose can be accurately detected.

(4) According to this embodiment, when the central processing section 26 determines that the glucose level in the subject 9 is a value in an abnormal state, the supply amount control section 49 supplies insulin to the subject 9 from the opening section 43b of the first needle section 43. Accordingly, even when an operator who confirms the glucose level is not present, the insulin pump 42 can supply insulin to the subject 9.

(5) According to this embodiment, the first needle section 43, the second needle section 7, and the third needle section 8 each have a smaller major axis than a needle having a cylindrical shape of the related art. Accordingly, even when the first needle section 43, the second needle section 7, and the third needle section 8 are stuck into the subject 9, the subject 9 can be made to feel less pain.

### Fourth Embodiment

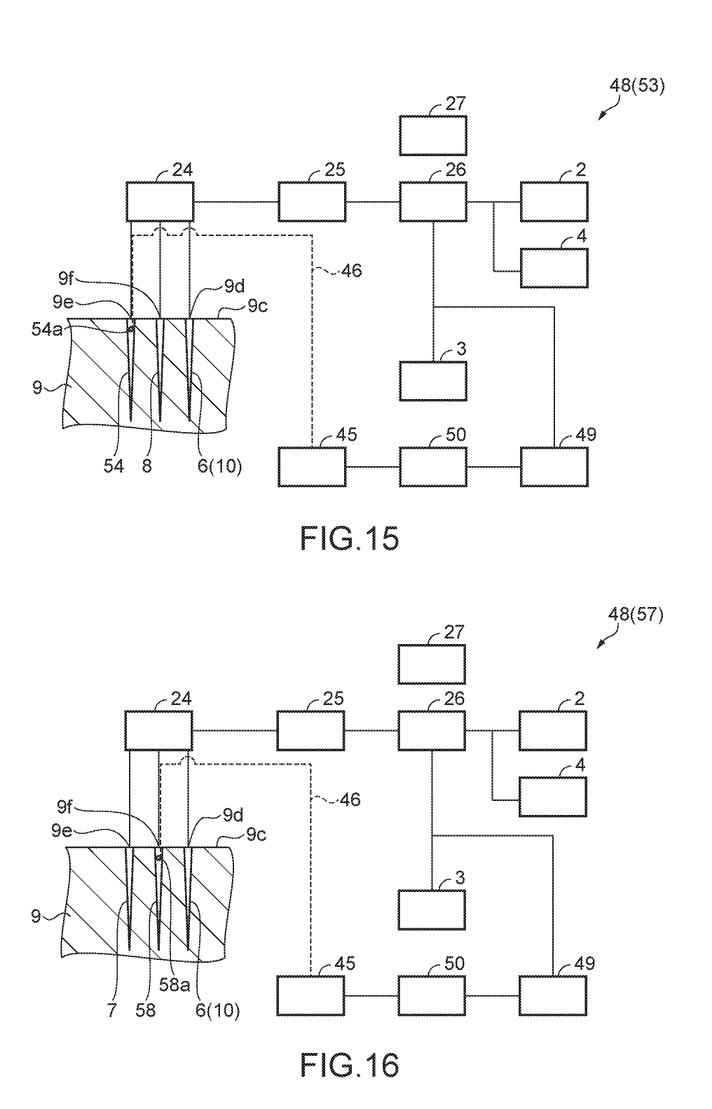

Next, one embodiment of an insulin pump will be described with reference to FIG. 15. FIG. 15 is a block diagram showing the configuration of a control section. This embodiment is different from the third embodiment in that insulin is supplied from a second needle section. Incidentally, the description of the same points as those of the third embodiment will be omitted.

That is, in this embodiment, as shown in FIG. 15, an insulin pump 53 as a drug solution supply device includes a control section 48 and a pump 45 in the same manner as in the third embodiment. Then, the insulin pump 53 includes a second needle section 54 as an injection needle, a second sensor electrode, and a counter electrode, and in the inside of the second needle section 54, a hole is placed. The hole is connected to an opening section 54a as a hole section placed on the side face of the second needle section 54. Then, a pipe 46 is connected to the hole in the inside of the second needle section 54. When the pump 45 is driven, insulin is discharged from a tank 44, passes through the pump 45, the pipe 46, and the hole in the inside of the second needle section 54, and is supplied to a subject 9 from the opening section 54a of the second needle section 54.

A first needle section 6 is inserted into the subject 9 by piercing the skin surface 9c at a first insertion place 9d. The second needle section 54 is inserted into the subject 9 by piercing the skin surface 9c at a second insertion place 9e. A third needle section 8 is inserted into the subject 9 by piercing the skin surface 9c at a third insertion place 9f.

When the detection film 10 reacts with glucose, an electric current flows between the detection film 10 and the second needle section 54. Then, by detecting the electric current by the electric current detection circuit 24, the glucose level in the interstitial fluid can be detected. Then, when the central processing section 26 determines that the glucose level in the subject 9 is a value in an abnormal state, the supply amount control section 49 supplies insulin to the subject 9 from the opening section 54a of the second needle section 54.

The second needle section 54 corresponds to an injection needle. One of the first needle section 6 and the third needle section 8 corresponds to a first electrode needle, and the other corresponds to a second electrode needle. Then, on the circumferential wall surface of each needle of the first electrode needle, the second electrode needle, and the injection needle, any one of a working electrode, a counter electrode, and a reference electrode is placed, and the working electrode, the counter electrode, and the reference electrode are placed on any of the first electrode needle, the second electrode needle, and the injection needle.

As described above, according to this embodiment, the following effect is obtained.

(1) According to this embodiment, the second needle section 54 functions as both a needle which supplies insulin and a needle of the counter electrode. Therefore, a pain spot of the subject 9 can be made less stimulated as compared with the case where a needle of the counter electrode is inserted into the subject 9 separately from the second needle section 54. Accordingly, the insulin pump 53 enables the subject 9 to feel less pain. In addition, according to this configuration, the same effects as the effects (1), (3), (4), and (5) of the third embodiment can be obtained.

### Fifth Embodiment

Next, one embodiment of an insulin pump will be described with reference to FIG. 16. FIG. 16 is a block diagram showing the configuration of a control section. This embodiment is different from the third embodiment in that insulin is supplied from a third needle section. Incidentally, the description of the same points as those of the third embodiment will be omitted.

That is, in this embodiment, as shown in FIG. 16, an insulin pump 57 as a drug solution supply device includes a control section 48 and a pump 45 in the same manner as in the third embodiment. Then, the insulin pump 57 includes a third needle section 58 as an injection needle and a reference electrode, and in the inside of the third needle section 58, a hole is placed. The hole is connected to an opening section 58a as a hole section placed on the side face of the third needle section 58. Then, a pipe 46 is connected to the hole in the inside of the third needle section 58. When the pump 45 is driven, insulin is discharged from a tank 44, passes through the pump 45, the pipe 46, and the hole in the inside of the third needle section 58, and is supplied to a subject 9 from the opening section 58a of the third needle section 58.

A first needle section 6 is inserted into the subject 9 by piercing the skin surface 9c at a first insertion place 9d. A second needle section 7 is inserted into the subject 9 by piercing the skin surface 9c at a second insertion place 9e. The third needle section 58 is inserted into the subject 9 by piercing the skin surface 9c at a third insertion place 9f.

When the detection film 10 reacts with glucose, an electric current flows between the detection film 10 and the second needle section 7. Then, by detecting the electric current by the electric current detection circuit 24, the glucose level in the interstitial fluid can be detected. Then, when the central processing section 26 determines that the glucose level in the subject 9 is a value in an abnormal state, the supply amount control section 49 supplies insulin to the subject 9 from the opening section 58a of the third needle section 58.

The third needle section 58 corresponds to an injection needle. One of the first needle section 6 and the second needle section 7 corresponds to a first electrode needle, and the other corresponds to a second electrode needle. Then, on the circumferential wall surface of each needle of the first electrode needle, the second electrode needle, and the injection needle, any one of a working electrode, a counter electrode, and a reference electrode is placed, and the working electrode, the counter electrode, and the reference electrode are placed on any of the first electrode needle, the second electrode needle, and the injection needle.

As described above, according to this embodiment, the following effect is obtained.

(1) According to this embodiment, the third needle section 58 functions as both a needle which supplies insulin and a needle of the reference electrode. Therefore, a pain spot of the subject 9 can be made less stimulated as compared with the case where a needle of the reference electrode is inserted into the subject 9 separately from the third needle section 58. Accordingly, the insulin pump 57 enables the subject 9 to feel less pain. In addition, according to this configuration, the same effects as the effects (1), (3), (4), and (5) of the third embodiment can be obtained.

### Sixth Embodiment

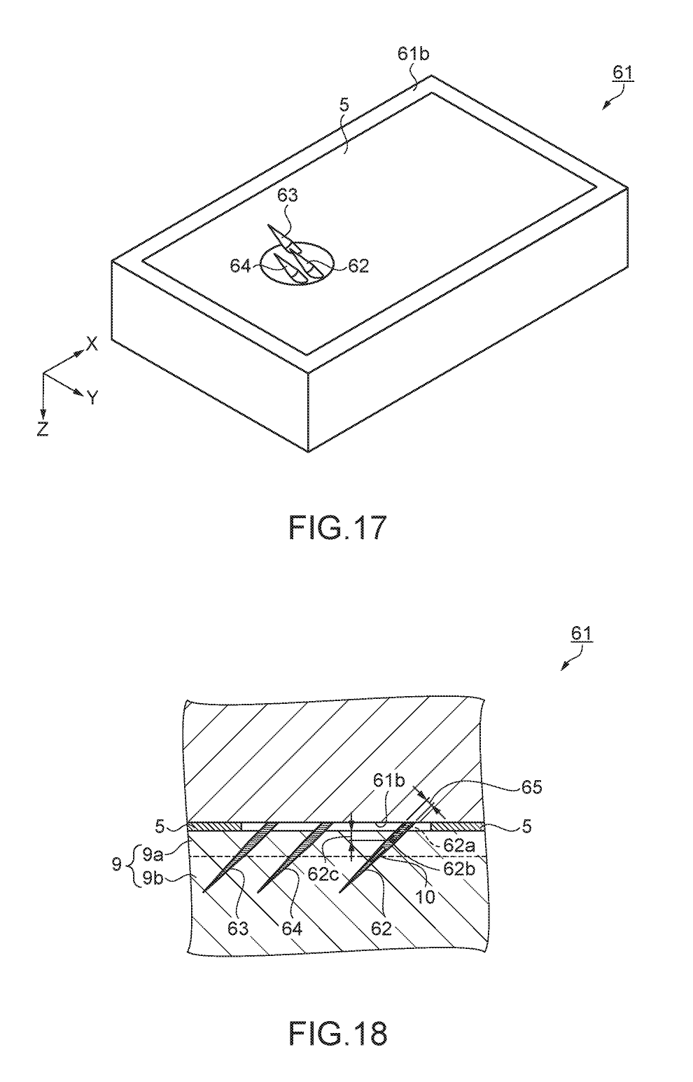

Next, one embodiment of an insulin pump will be described with reference to FIGS. 17 and 18. FIG. 17 is a schematic perspective view showing the structure of an insulin pump, and is a view seen from the rear side of an insulin pump 61 as a drug solution supply device. FIG. 18 is a schematic side cross-sectional view showing the structures of a first needle section to a third needle section, and is a view showing that the first needle section to the third needle section are stuck into a subject 9. This embodiment is different from the third embodiment in that the first needle section to the third needle section are stuck obliquely with respect to the skin surface. Incidentally, the description of the same points as those of the third embodiment will be omitted.

That is, in this embodiment, as shown in FIG. 17, on the rear face 61b of the insulin pump 61, a first needle section 62 as a first sensor electrode and a working electrode, a second needle section 63 as a second sensor electrode and a counter electrode, and a third needle section 64 as a reference electrode are placed. The first needle section 62, the second needle section 63, and the third needle section 64 are placed obliquely with respect to the rear face 61b and are placed parallel to one another.

As shown in FIG. 18, the first needle section 62 has a shape such that one end of a cylinder is in the shape of a cone, and the major axis 65 of the first needle section is 0.1 mm or more and 0.3 mm or less. Therefore, the first needle section 62 of the insulin pump 61 can be made hard to break and enables the subject 9 to feel less pain. A detection film 10 is placed on the first needle section 62 from the center to the tip end side. Then, in the inside of the first needle section 62, a hole 62a is placed, and the hole 62a is connected to an opening section 62b as a hole section located between the root of the first needle section 62 and the detection film 10.

When the pump 45 is driven, insulin is discharged from a tank 44, passes through a pump 45, a pipe 46, and the hole 62a, and flows out from the opening section 62b of the first needle section 62 to the subject 9. The distance 62c between the center of the opening section 62b and the rear face 61b is set within the range of 1 mm or more and 2 mm or less. At this time, the opening section 62b is located in a skin tissue 9a, and therefore, insulin can be supplied to the skin tissue 9a. Then, a glucose level in a hypodermal tissue 9b can be measured. Interstitial fluid is present more in the hypodermal tissue 9b than in the skin tissue 9a. There are more chances for glucose to come in contact with the detection film 10 when the detection film 10 is located in a place where interstitial fluid is present more, and therefore, a glucose level can be more accurately measured.

The second needle section 63 and the third needle section 64 each have a shape such that one end of a cylinder is in the shape of a cone, and the major axes of the second needle section 63 and the third needle section 64 are each 0.1 mm or more and 0.3 mm or less. Therefore, the second needle section 63 and the third needle section 64 of the insulin pump 61 can be made hard to break and enables the subject 9 to feel less pain. Further, the first needle section 62, the second needle section 63, and the third needle section 64 are inserted obliquely into the subject 9. According to this, when a force in the normal direction of the rear face 61b acts on the insulin pump 61, the direction of the force and the longitudinal direction of each needle intersect each other. Therefore, the skin tissue 9a acts so that the first needle section 62, the second needle section 63, and the third needle section 64 are not pulled out. As a result, even if an external force is applied to the insulin pump 61, the insulin pump 61 can be made difficult to separate from the subject 9.

The first needle section 62 corresponds to an injection needle. One of the second needle section 63 and the third needle section 64 corresponds to a first electrode needle, and the other corresponds to a second electrode needle. Then, on the circumferential wall surface of each needle of the first electrode needle, the second electrode needle, and the injection needle, any one of the working electrode, the counter electrode, and the reference electrode is placed, and the working electrode, the counter electrode, and the reference electrode are placed on any of the first electrode needle, the second electrode needle, and the injection needle.

Incidentally, the present embodiment is not limited to the above-mentioned embodiments, and a person of ordinary skill in the art can add various changes and improvements within the technical ideas of the invention. Hereinafter, modifications will be described.

### First Modification

In the above first embodiment, on the first needle section 6, the base layer 12, the ITO electrode layer 13, and the enzyme electrode layer 14 are placed. However, the base layer 12 and the ITO electrode layer 13 may be omitted, and the first needle section 6 may be used as part of a wire. Since the step of placing the base layer 12 and the ITO electrode layer 13 can be omitted, the measuring device 1 can be produced with high productivity. Further, by forming the first needle section 6 using the material of the enzyme electrode layer 14, the first needle section 6 may be allowed to function also as the enzyme electrode layer 14. Since the step of placing the enzyme electrode layer 14 can be omitted, the measuring device 1 can be produced with high productivity.

### Second Modification

In the above first embodiment, the first needle section 6 to the third needle section 8 each have a conical shape. However, the first needle section 6 to the third needle section 8 may each be formed into a shape such that one end of a cylinder is in the shape of a cone in the same manner as in the sixth embodiment. According to this, the shape of a portion having a conical shape can be adjusted.

### Third Modification

In the above first embodiment, the first needle section 6 to the third needle section 8 each extend in the normal direction of the rear face 1b. However, also in the measuring device 1, in the same manner as in the sixth embodiment, the first needle section 6 to the third needle section 8 may each extend obliquely with respect to the normal direction of the rear face 1b. The measuring device 1 can be made difficult to separate from the subject 9.

### Fourth Modification

In the above first embodiment, platinum is used as the material of the second needle section 7, and the second needle section 7 is used as a counter electrode. However, an insulating material may be used as the material of the second needle section 7, and a platinum film may be placed on the surface of the second needle section 7. Also in this case, the second needle section 7 can be used as a counter electrode. Similarly, silver is used as the material of the third needle section 8, and the third needle section 8 is used as a reference electrode. However, an insulating material may be used as the material of the third needle section 8, and a silver film may be placed on the surface of the third needle section 8. Also in this case, the third needle section 8 can be used as a reference electrode. Incidentally, the contents of the first modification to the fourth modification may be applied to the second embodiment to the fifth embodiment.

### Fifth Modification

In the above first embodiment, a film which detects a glucose level is used as the detection film 10. However, by changing the enzyme, a component other than glucose may be detected. For example, the level of uric acid, urea, or total amylase may be detected. This content may be applied to the second embodiment. Further, this content may be applied to the third embodiment to the sixth embodiment, and a given drug solution may be supplied.

### Sixth Modification

In the above first embodiment, the first needle section 6 to the third needle section 8 are placed in the measuring device 1. Then, in the above second embodiment, the first needle section 6 and the second needle section 7 are placed in the measuring device 35. However, also in a measuring device in which four or more needle sections are placed, each needle may be used as an electrode. Also in this case, the major axis of each needle can be made short, and therefore, a device which less stimulates a pain spot of the subject 9 can be provided.

### Seventh Modification

In the above first embodiment, the measuring device 1 includes the speaker 4. However, other than this, in the measuring device 1, a vibrating device may be placed. Then, when it is determined that the glucose level is a value in an abnormal state, the vibrating device may be allowed to vibrate. Even in an environment where a warning sound cannot be output, the subject 9 can be notified that the glucose level is a value in an abnormal state.

### Eighth Modification

In the above first embodiment, the first needle section 6 to the third needle section 8 are disposed at the vertices of an isosceles triangle. Then, the distance between the first needle section 6 and the third needle section 8 is shorter than the distance between the first needle section 6 and the second needle section 7, and also shorter than the distance between the second needle section 7 and the third needle section 8. However, the disposition of the first needle section 6 to the third needle section 8 is not limited thereto. The first needle section 6 to the third needle section 8 may be disposed linearly. Further, the first needle section 6 to the third needle section 8 may be disposed at the vertices of a triangle in which all sides are of different length. Also in this case, it is preferred that the distance between the first needle section 6 and the third needle section 8 is shorter than the distance between the first needle section 6 and the second needle section 7, and also shorter than the distance between the second needle section 7 and the third needle section 8. Thus, the increase in the resistance between the first needle section 6 and the third needle section 8 can be accurately detected. Further, when the electric current detection circuit 24 can accurately detect the increase in the resistance between the first needle section 6 and the third needle section 8, the first needle section 6 to the third needle section 8 may be disposed at the vertices of an equilateral triangle. Incidentally, this content may also be applied to the above third embodiment to the above sixth embodiment.

### Ninth Embodiment

In the above third embodiment, the central processing section 26 outputs an instruction signal to discharge insulin and the data of the glucose level to the supply amount control section 49. Then, the supply amount control section 49 calculates the amount of insulin to be supplied to the subject 9. However, it is not limited thereto, and the central processing section 26 may calculate the amount of insulin to be supplied to the subject 9. Then, the central processing section 26 may output an instruction signal to discharge insulin and the data of the amount of insulin to be supplied to the subject 9 to the supply amount control section 49. By integrating the calculation function in the central processing section 26, the renewal of the calculation function can be easily performed.

The entire disclosure of Japanese Patent Application No. 2015-145573 filed Jul. 23, 2015 is expressly incorporated by reference herein.

What is claimed is:

1. A biological information measuring device, comprising:
a first sensor electrode which is inserted into a body by piercing the skin surface;
a second sensor electrode which is inserted into the body by piercing the skin surface; and
an electric current detection section which detects an electric current between the first sensor electrode and the second sensor electrode, wherein
the device measures in-vivo information.

2. The biological information measuring device according to claim 1, wherein the major axis of the first sensor electrode is 0.1 mm or more and 0.3 mm or less.

3. The biological information measuring device according to claim 1, wherein the straight-line distance between the first sensor electrode and the second sensor electrode is 1 mm or more and 50 mm or less.

4. The biological information measuring device according to claim 1, wherein the first sensor electrode is provided with a sensing detection layer.

5. The biological information measuring device according to claim 1, wherein
the in-vivo information is information associated with glucose,
the first sensor electrode is a working electrode which is provided with an enzyme layer as the sensing detection layer, and
the second sensor electrode is a counter electrode which receives an electric current generated in the first sensor electrode.

6. The biological information measuring device according to claim 1, wherein
the biological information measuring device further comprises a reference electrode which is used for detecting a resistance to an electric current flowing through the first sensor electrode, and
the straight-line distance between the first sensor electrode and the reference electrode is shorter than the straight-line distance between the first sensor electrode and the second sensor electrode.

7. The biological information measuring device according to claim 6, wherein the straight-line distance between the first sensor electrode and the reference electrode is 1 mm or more and 50 mm or less.

8. The biological information measuring device according to claim 6, wherein the straight-line distance between the reference electrode and the second sensor electrode is 1 mm or more and 50 mm or less.

9. The biological information measuring device according to claim 5, wherein the counter electrode contains platinum.

10. The biological information measuring device according to claim 5, wherein a resin film is placed on the working electrode and the counter electrode.

11. A drug solution supply device, comprising:
a working electrode;
a counter electrode;
a reference electrode;
a biological information measuring section which measures in-vivo information by applying a predetermined voltage to the reference electrode and detecting an electric current between the working electrode and the counter electrode;
a first electrode needle which is inserted into a body by piercing the skin surface;
a second electrode needle which is inserted into the body by piercing the skin surface; and
an injection needle which is inserted into the body by piercing the skin surface and injects a drug solution into the body, wherein
on the circumferential wall surface of each needle of the first electrode needle, the second electrode needle, and the injection needle, any one of the working electrode, the counter electrode, and the reference electrode is placed, and the working electrode, the counter electrode, and the reference electrode are placed on any of the first electrode needle, the second electrode needle, and the injection needle.

12. The drug solution supply device according to claim 11, wherein
the in-vivo information is information associated with glucose, and
the drug solution is insulin.
